(19) United States
(12) Patent Application Publication
(10) Pub. No.: US 2015/0273070 A1
(43) Pub. Date: Oct. 1, 2015

(54) MODIFIED RELEASE OF 4-METHYL-3-[[4-(3-PYRIDINYL)-2-PYRIMIDINYL]AMINO]-N-[5-(4-METHYL-1H-IMIDAZOL-1-YL)-3-(TRIFLOUOROMETHYL)PHENYL] BENZAMIDE SOLUBILIZED USING ORGANIC ACIDS

(71) Applicants: Shoufeng Li, Basking Ridge, NJ (US); Saran Kumar, Edison, NJ (US); Nikhil Javant Kavimandan, South Plainfield, NJ (US); Enxian Lu, Morris Plains, NJ (US)

(72) Inventors: Shoufeng Li, Basking Ridge, NJ (US); Saran Kumar, Edison, NJ (US); Nikhil Javant Kavimandan, South Plainfield, NJ (US); Enxian Lu, Morris Plains, NJ (US)

(73) Assignee: Novartis AG, Basel (CH)

(21) Appl. No.: 14/126,219

(22) PCT Filed: Jun. 13, 2012
(86) PCT No.: PCT/US2012/042205

§ 371 Date: Dec. 13, 2013

Related U.S. Application Data
(60) Provisional application No. 61/496,913, filed on Jun. 14, 2011.
(60) Provisional application No. 61/541,306, filed on Sep. 30, 2011.

(51) Int. Cl. A61K 47/22; A61K 31/506; A61K 47/12; C07D 401/14

(52) U.S. Cl. CPC A61K 47/22; C07D 401/14; A61K 31/506; A61K 47/12

(57) ABSTRACT

Soluble pharmaceutical compositions of nilotinib or a pharmaceutically acceptable salt thereof were invented using one or more organic acids that function as a solubilizing agent, increasing the bioavailability of nilotinib and supressing the food effect associated with certain compositions of nilotinib. The pharmaceutical compositions are in the form of solid oral dosage forms, including capsules and tablets.

### PRIORITY

1. Field of the Invention

The present invention relates to a pharmaceutical composition comprising a therapeutic compound of nilotinib (Formula I) that is present in a solubilized or amorphous state. Such a pharmaceutical composition further comprises at least one organic acid which functions as a solubilizing agent, increasing the bioavailability of nilotinib and suppressing the food effect associated with certain compositions of nilotinib.

2. Background of the Invention

Nilotinib is 4-Methyl-3-[[4-(3-pyridinyl)-2-pyrimidinyl]amino]-N[5-(4-methyl-1H-imidazol-1-yl)-3-(trifluoromethyl)phenyl] benzamide. A particularly useful salt of nilotinib is nilotinib hydrochloride monohydrate. These therapeutic compounds have utility as inhibitors of the protein tyrosine kinase (TK) activity of Bcr-Abl. Examples of conditions that may be treated by such therapeutic compounds include, but are not limited to, chronic myeloid leukemia and gastrointestinal stromal tumors.

There is a need to formulate nilotinib and the other therapeutic compounds hereinafter disclosed into pharmaceutical compositions, especially solid oral dosage forms, such that the therapeutic benefits of the compounds may be delivered to a patient in need thereof. One problem to providing such compositions including nilotinib is the physiochemical properties of nilotinib, since nilotinib and its salts are poorly water soluble compounds and are difficult to formulate and deliver (i.e., made bioavailable when ingested orally).

### SUMMARY OF THE INVENTION

The present invention provides solublized or amorphous pharmaceutical compositions of nilotinib or a pharmaceutical acceptable salt thereof using one or more organic acids that function as a solubilizing agent, increasing the bioavailability of nilotinib and suppressing the food effect associated with certain compositions of nilotinib. The pharmaceutical compositions are in the form of oral dosage forms, preferably solid oral dosage forms, including capsules, tablets and multiparticulates.

The aspects, advantageous features and preferred embodiments of the present invention summarized in the following items, respectively alone or in combination, relating to the invention:

An amorphous 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof.

A dosage form comprising amorphous 4-Methyl-3-[[4-(3-pyrldinyl)-2-pyrimidinyl]amino]-N-[5-(4-methyl-1H-imidazol-1-yl)-3-(trifluoromethyl)phenyl] benzamide or a pharmaceutically acceptable salt thereof.

A dosage form of item 2 comprising 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and at least one organic acid.

A dosage form of item 2 or 3 comprising 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and at least one organic acid, having a fasted state bioavailability that exceeds 130% of marketed Tasigna™ hard-gelatin capsule.

A dosage form of any one of items 3 to 5 comprising 4-Methyl-3-[[4-(3pyridinyl)-2-pyrimldinyl]amino]-N-[5-(4-methyl-1H-imidazol-1-yl)-3-(trifluoromethyl)phenyl] benzamide or a pharmaceutically acceptable salt thereof and at least one organic acid, having a fed/fasted ratio of 0.8-1.5 for AUC and/or Cₘₐₓ.

The dosage form of any one of items 3 to 6, wherein said at least one organic acids is selected from acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, glutamic acid, aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid and ascorbic acid.

The dosage form, wherein the organic acid is citric acid.

The dosage form, wherein the organic acid is lactic acid.

The dosage form, wherein the organic acid is acetic acid.

The dosage form, further comprising a surfactant or an anionic polymer.

The dosage form, wherein the surfactant or the anionic polymer is CYP3A4 or Pg-P inhibitor.

The dosage form, wherein the surfactant is Poloxamer 407 and/or Vitamin E TPGS.

The dosage form, wherein the polymer is Eudragid L100-55.

The dosage form, wherein the dosage form has water content of less than 10% w/w, preferably less than 5% w/w, particularly less than 2% w/vv.

The dosage form, further comprising excipients for solidifying the dosage form.

The dosage form, wherein the dosage form is soiid.

The dosage form, wherein the dosage form is a tablet.

The dosage form, wherein the dosage form is a capsule.

A method for preparing amorphous 4-Methyl-3-[[4-(3-pyridinyl)-2-pyrimidinyl]amino]-N-[5-(4-methyl-1H-imidazol-1-yl)-3-(trifluoromethyl)phenyl]benzamide or a pharmaceutically acceptable salt thereof, comprising the step of adding at least one organic acid.

A method for preparing a dosage form comprising amorphous 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and at least one organic acid, comprising the step of melt extruding 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and the at least one organic acid.

A method, wherein 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and at least one organic acid are mixed and melt extruded together.

A method of preparing a dosage form comprising 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and at least one organic acid comprising the step of spray drying at least partly dissolved of 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and adding the at least one organic acid.

The method, wherein the 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and the at least one organic acid together are in a solution or suspension for spray drying.

The method of any one of items, further comprising a step of adding a surfactant or an anionic polymer.

The method, wherein the surfactant or the anionic polymer is CYP3A4 or Pg-P inhibitor.

The method, wherein the surfactant is Poloxamer 407 and/or Vitamin E TPGS.

The method, wherein the polymer is Eudragid L100-55.

The methods, comprising a further step of obtaining a solid dosage form.

The method, wherein the solid dosage form is a tablet or a capsule.

Use of organic acid for increasing of bioavailability of nilotinib.

Use of organic acid for suppressing the food effect associated with pharmaceutical composition comprising nilotinib or a pharmaceutically acceptable salt thereof.

A dosage form of any one of items—for use as a medicine.

The dosage form, wherein the medicine is stored under refrigeration at 2 to 8° C.

### BRIEF DESCRIPTION OF THE DRAWINGS

FIG. 1 summarizes the dissolution profile for a nilotinib lactic acid formulation.

FIG. 2 summarizes Cmax data for a nilotinib lactic acid formulation tested in dogs.

FIG. 3 summarizes AUC data for a nilotinib lactic acid formulation tested in dogs.

FIG. 4 summarizes X-ray diffraction (XRD) data for a nilotinib citric acid intermediate.

FIG. 5 summarizes differential scanning calorimetric data for a nilotinib citric acid intermediate.

FIG. 6 summarizes thermogravimmetric data for a nilotinib citric acid intermediate.

FIG. 7 summarizes thermogravimmetric data for a nilotinib citric acid intermediate.

FIG. 8 summarizes XRD data for a nilotinib citric acid formulation after 6 month storage at ambient condition.

FIG. 9 summarizes the two-step dissolution profile for a nilotinib citric acid formulation.

FIG. 10 summarizes the two-step dissolution profile for a nilotinib citric acid MR tablet (slow).

FIG. 11 summarizes Cₘₐₓ data for a nilotinib citric acid formulation tested in dogs.

FIG. 12 summarizes AUC data for a nilotinib citric acid formulation tested in dogs.

### DETAILED DESCRIPTION OF THE INVENTION

The present invention provides solublized or amorphous pharmaceutical compositions of nilotinib or a pharmaceutically acceptable salt thereof using one or more organic acids that function as a solubilizing agent, increasing the bioavailability of nilotinib and supressing the food effect associated with certain compositions of nilotinib.

The soluble solid dosage forms of nilotinib are subsequently encapsulated into hard gelatin capsules, compressed into tablets, or filled into sachets to form solid oral dosage forms.

As used herein, nilotinib refers to 4-Methyl-3-[[4-(3-pyrldinyl)-2-pyrimidinyl]amino]-N-[5-(4-methyl-1H-imidazol-1-yl)-3-(trifluoromethyl)phenyl] benzamide of formula I:

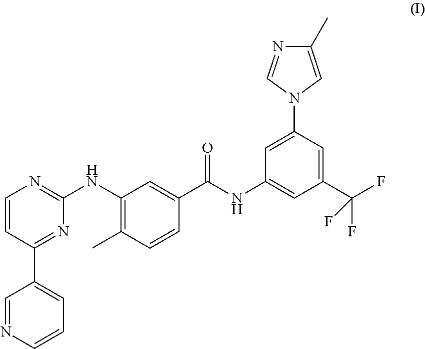 [SMILES: Cc1cn(-c2cc(NC(=O)c3ccc(C)c(Nc4nccc(-c5cccnc5)n4)c3)cc(C(F)(F)F)c2)cn1]

Nilotinib is a member of compounds of formula (II)

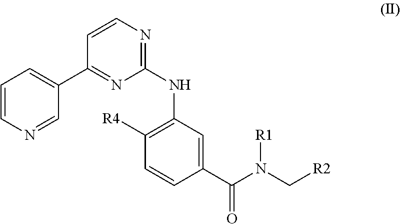

wherein
R₁ represents hydrogen, lower alkyl, lower alkoxy-lower alkyl, acyloxy-lower alkyl, carboxy-lower alkyl, lower alkoxycarbonyl-lower alkyl, or phenyl-lower alkyl;
R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;
and R₃ represents hydroxy, lower alkoxy, acyloxy, carboxy, lower alkoxycarbonyl, carbamoyl, N-mono- or N,N-disubstituted carbamoyl, amino, mono- or disubstituted amino, cycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;
or wherein R₁ and R₂ together represent alkylene with four, five or six carbon atoms optionally mono- or disubstituted by lower alkyl, cycloalkyl, heterocyclyl, phenyl, hydroxy, lower alkoxy, amino, mono- or disubstituted amino, oxo, pyridyl, pyrazinyl or pyrimidinyl; benzalkylene with four or five carbon atoms; oxaalkylene with one oxygen and three or four carbon atoms; or azaalkylene with one nitrogen and three or four carbon atoms wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-disubstituted carbamoyl-lower alkyl cycloalkyl, lower alkoxycarbonyl, carboxy, phenyl, substituted phenyl, pyridinyl, pyrimidinyl, or pyrazinyl;
R₄ represents hydrogen, lower alkyl, or halogen;
and a N-oxide and to the pharmaceutically acceptable salts of such a compound.

Such therapeutic compounds are suitable for the preparation of a pharmaceutical composition for the treatment of kinase dependent diseases, especially Bcr-Abl and Tie-2kinase dependent diseases, for example, as drugs to treat one or more proliferative diseases.

Within the definition of “therapeutic compound,” the prefix “lower” denotes a radical having up to and including a maximum of seven, especially up to and including a maximum of four carbon atoms, the radicals in question being either linear or branched with single or multiple branching.

As used herein, where the plural form is used for compounds, salts, and the like, this is taken to mean also a single compound, salt, or the like.

Any asymmetric carbon atoms may be present in the (R)-, (S)- or (R,S)-configuration. for example in the (R)- or (S)-configuration. The compounds may thus be present as mixtures of isomers or as pure isomers, for example as enantiomer-pure diastereomers. Also contemplated within the present invention is the use of any possible tautomers of the compounds of formula I.

Lower alkyl is for example alkyl with from and including one up to and including seven, for example from and including one to and including four, and is linear or branched; for example, lower alkyl is butyl, such as n-butyl, sec-butyl, isobutyl, tert-butyl, propyl, such as n-propyl or isopropyl, ethyl or methyl. For example lower alkyl is methyl, propyl or tert-butyl.

Lower acyl is for example formyl or lower alkylcarbonyl, in particular acetyl.

An aryl group is an aromatic radical which is bound to the molecule via a bond located at an aromatic ring carbon atom of the radical, in an exemplary embodiment, aryl is an aromatic radical having six to fourteen carbon atoms, especially phenyl, naphthyl, tetrahydronaphthyl, fluorenyl or phenanthrenyl, and is unsubstituted or substituted by one or more, for example up to three, especially one or two substituents, especially selected from amino, mono- or disubstituted amino, halogen, lower alkyl, substituted lower alkyl, lower alkenyl, lower alkynyl, phenyl, hydroxy, etherified or esterified hydroxy, nitro, cyano, carboxy, esterified carboxy, alkanoyl, benzoyl, carbamoyl, N-mono- or N,N-disubstituted carbamoyl, amidino, guanidino, ureido, mercapto, sulfo, lower alkylthio, phenylthio, phenyl-lower alkylthio, lower alkylphenylthio, lower alkylsulfinyl, phenylsulfinyl, phenyl-lower alkylsulfinyl, lower alkylphenylsulfinyl, lower aikyfsulfonyl, phenyisulfonyl, phenyl-lower alkylsulfonyl, lower alkylphenylsulfonyl, halogen-lower alkylmercapto, halogen-lower alkylsulfonyl, such as especially triflucromethanesulfonyl, dihydroxybora (—B(OH)2), heterocyclyl, a mono- or bicyclic heteroaryl group and lower alkylene dioxy bound at adjacent C-atoms of the ring, such as methylene dioxy. Aryl is for example phenyl, naphthyl or tetrahydronaphthyl, which in each case is either unsubstituted or independently substituted by one or two substituents selected from the group comprising halogen, especially fluorine, chlorine, or bromine; hydroxy; hydroxy etherified by lower alkyl, e.g. by methyl, by halogen-lower alkyl, e.g. trifluoromethyl, or by phenyl; lower alkylene dioxy bound to two adjacent C-atoms, e.g. methylenedioxy, lower alkyl, e.g. methyl or propyl; halogen-lower alkyl, e.g. trifluoromethyl; hydroxy-lower alkyl, e.g. hydroxymethyl or 2-hydroxy-2-propyl; lower alkoxy-lower alkyl; e.g. methoxymethyl or 2-methoxyethyl; lower alkoxycarbonyl-lower alkyl. e.g. methoxy-carbonylmethyl; lower alkynyl, such as 1-propynyl; esterified carboxy, especially lower alkoxycarbonyl, e.g. methoxycarbonyl, n-propoxy carbonyl or iso-propoxy carbonyl; N-mono-substituted carbamoyl, in particular carbamoyl monosubstituted by lower alkyl, e.g. methyl, n-propyl or iso-propyl; amino; lower alkylamino, e.g. methylamino; di-lower alkylamino, e.g. dimethylamino or diethylamino; lower alkylene-amino, e.g. pyrrolidine or piperidino; lower oxaalkylene-amino, e.g. morpholino, lower azaalkylene-amino, e.g. piperazino, acylamino, e.g. acetylamino or benzoylamino; lower alkylsulfonyl, e.g. methylsulfonyl; sulfamoyl; or phenylsulfonyl.

A cycloalkyl group is for example cyclopropyl, cyclopentyl, cyclohexyl or cycloheptyl, and may be unsubstituted or substituted by one or more, especially one or two, substitutents selected from the group defined above as substituents for aryl, e.g., by lower alkyl, such as methyl, lower alkoxy, such as methoxy or ethoxy, or hydroxy, and further by oxo or fused to a benzo ring, such as in benzcyclopentyl or benzcyclohexyl.

Substituted alkyl is alkyl as last defined, especially lower alkyl, for example methyl; where one or more, especially up to three, substituents may be present, primarily from the group selected from halogen, especially fluorine, amino, N-lower alkylamino, N,N-di-lower alkylamino, N-lower alkanoylamino, hydroxy, cyano, carboxy, lower alkoxycarbonyl, and phenyl-lower alkoxycarbonyl. Trifluoromethyl is especially useful.

Mono- or disubstituted amino is especially amino substituted by one or two radicals selected independently of one another from lower alkyl, such as methyl; hydroxy-lower alkyl, such as 2-hydroxyethyl; lower alkoxy lower alkyl, such as methoxy ethyl; phenyl-lower alkyl, such as benzyl or 2-phenylethyl; lower alkanoyl, such as acetyl; benzoyl; substituted benzoyl, wherein the phenyl radical is especially substituted by one or more, for example one or two, substituents selected from nitro, amino, halogen, N-lower alkylamlno, N,N-di-lower alkylamino, hydroxy, cyano, carboxy, lower alkoxycarbonyl, lower alkanoyl, and carbamoyl; and phenyl-lower alkoxycarbonyl, wherein the phenyl radical is unsubstituted or especially substituted by one or more, for example one or two, substituents selected from nitro, amino, halogen, N-lower alkylamino, N,N-di-lower alkylamino, hydroxy, cyano, carboxy, lower alkoxycarbonyl, lower alkanoyl, and carbamoyl; and is for example N-lower alkylamino, such as N-methylamino, hydroxy-lower alkylamino, such as 2-hydroxyethylamino or 2-hydroxypropyl, lower alkoxy lower alkyl, such as methoxy ethyl, phenyl-lower alkylamino, such as benzylamino, N,N-di-lower alkylamino, N-phenyl-lower alkyl-N-lower alkylamino, N,N-di-lower alkylphenylamino, lower alkanoylamino, such as acetylamino, or a substituent selected from the group comprising benzoylamino and phenyl-lower alkoxycarbonylamino, wherein the phenyl radical in each case is unsubstituted or especially substituted by nitro or amino, or also by halogen, amino, N-lower alkylamino, N,N-di-lower alkylamino, hydroxy, cyano, carboxy, lower alkoxycarbonyl, lower alkanoyl, carbamoyl or aminocarbonylamino. Disubstituted amino is also lower alkylene-amino, e.g. pyrrolidine, 2-oxopyrrolidino or piperidino; lower oxaalkylene-amino, e.g. morpholino, or lower azaalkylene-amino, e.g. piperazino or N-substituted piperazino, such as N-methylpiperazino or N-methoxycarbonylpiperazino.

Halogen is especially fluorine, chlorine, bromine, or iodine, especially fluorine, chlorine, or bromine.

Etherified hydroxy is especially C₈-C₂₀alkyloxy, such as n-decyloxy, lower alkoxy, such as methoxy, ethoxy, isopropyloxy, or tert-butyloxy, phenyl-lower alkoxy, such as benzyloxy, phenyloxy, halogen-lower alkoxy, such as trifluoromethoxy, 2,2,2-trifluoroethoxy or 1,1,2,2-tetrafluoroethoxy, or lower alkoxy which is substituted by mono- or bicyclic hetero-aryl comprising one or two nitrogen atoms, for example lower alkoxy which is substituted by imidazolyl, such as 1H-imidazol-1-yl, pyrrolyl, benzimidazolyl, such as 1-benzimidazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, indolyl or thiazolyl.

Esterified hydroxy is especially lower alkanoyloxy, benzoyloxy, lower alkoxycarbonyloxy, such as tert-butoxycarbonyloxy, or phenyl-lower alkoxycarbonyloxy, such as benzyloxycarbonyloxy.

Esterified carboxy is especially lower alkoxycarbonyl, such as tert-butoxycarbonyl, iso-propoxycarbonyl, methoxycarbonyl or ethoxycarbonyl, phenyl-lower alkoxycarbonyl, or phenyloxycarbonyl.

Alkanoyl is primarily alkylcarbonyl, especially lower alkanoyl, e.g. acetyl.

N-Mono- or N,N-disubstituted carbamoyl is especially substituted by one or two substituents independently selected from lower alkyl, phenyl-lower alkyl and hydroxy-lower alkyl, or lower alkylene, oxa-lower alkylene or aza-lower alkylene optionally substituted at the terminal nitrogen atom.

A mono- or bicyclic heteroaryl group comprising zero, one, two or three ring nitrogen atoms and zero or one oxygen atom and zero or one sulfur atom, which groups in each case are unsubstituted or mono- or polysubstituted, refers to a heterocyclic moiety that is unsaturated in the ring binding the heteroaryl radical to the rest of the molecule in formula I and is for example a ring, where in the binding ring, but optionally also in any annealed ring, at least one carbon atom is replaced by a heteroatom selected from the group consisting of nitrogen, oxygen and sulfur; where the binding ring for example has five to twelve, e.g., five or six ring atoms; and which may be unsubstituted or substituted by one or more, especially one or two, substitutents selected from the group defined above as substitutents for aryl, most for example by lower alkyl, such as methyl, lower alkoxy, such as methoxy or ethoxy, or hydroxy. For example the mono- or bicyclic heteroaryl group is selected from 2H-pyrrolyl, pyrrolyl, imidazolyl, benzimidazolyl, pyrazolyl, indazolyt, purinyl, pyridyl, pyrazinyl, pyrimidinyl, pyridazinyl, 4H-quinolizinyl, isoquinolyl, quinoiyl, phthalazinyl, naphthyridinyl, quinoxalyl, quinazolinyl, quinnolinyl, pteridinyl, indolizinyl, 3H-indolyl, indolyl, isoindolyl, oxazolyl, isoxazolyl, thiazolyl, isothiazolyl, triazolyl, tetrazolyl, furazanyl, benzo[d]pyrazolyl, thienyl and furanyl. For example the mono- or bicyclic heteroaryl group is selected from the group consisting of pyrrolyl, imidazolyl, such as 1H-imidazol-1-yl, benzimidazolyl, such as 1-benzimidazolyl, indazolyl, especially 5-indazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, especially 4- or 8-quinolinyl, indolyl, especially 3-indolyl, thiazolyl, benzo[d]pyrazolyl, thienyl, and furanyl. In one exemplary embodiment of the invention the pyridyl radical is substituted by hydroxy in ortho position to the nitrogen atom and hence exists at least partially in the form of the corresponding tautomer which is pyridin-(1H)2-one. In another exemplary embodiment, the pyrimidinyl radical is substituted by hydroxy both in position 2 and 4 and hence exists in several tautomeric forms, e.g. as pyrimidine-(1H, 3H)2,4-dione.

Heterocyclyl is especially a five, six or seven-membered heterocyclic system with one or two heteroatoms selected from the group comprising nitrogen, oxygen, and sulfur, which may be unsaturated or wholly or partly saturated, and is unsubstituted or substituted especially by lower alkyl, such as methyl, phenyl-lower alkyl, such as benzyl, oxo, or heteroaryl, such as 2-piperazinyl; heterocyclyl is especially 2- or 3-pyrrolidinyl, 2-oxo-5-pyrrolidinyl, piperidinyl, N-benzyl-4-piperidinyl, N-lower alkyl-4-piperidinyl, N-lower alkyl-piperazinyl, morpholinyl, e.g. 2- or 3-morpholinyl, 2-oxo-1 H-azepin-3-yl, 2-tetrahydrofuranyl, or 2-methyl-1,3-dioxolan-2-yl.

Salts are especially the pharmaceutically acceptable salts of compounds of formula I. Such salts are formed, for example, as acid addition salts, for example with organic or inorganic acids, from compounds of formula I with a basic nitrogen atom, especially the pharmaceutically acceptable salts. Suitable inorganic acids include, but are not limited to, halogen acids, such as hydrochloric acid, sulfuric acid, or phosphoric acid.

Suitable organic acids are, for example, carboxylic, phosphoric, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3-or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

According to one embodiment, a pharmaceutical composition comprises nilotinib or a pharmaceutically acceptable salt thereof and one or more organic acids that function as a solubilizing agent, increasing the bioavailability of nilotinib and supressing the food effect associated with certain compositions of nilotinib. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl- N-ethyl- or N-propyl-sulfamic acid, or other organic protonic acids, such as ascorbic acid.

One useful salt of nilotinib is nilotinib hydrochloride monohydrate, or 4-Methyl-N-[3-(4-methyl-1H-imidazol-1-yl)-5-(trifluromethyl)phenyl]-3-[(4-pyridine-3ylpyrimidin-2-yl)amino]benzamide hydrochloride hydrate. Suitable salts of nilotinib and polymorphs thereof are disclosed in more general in WO2007/015870 and WO2007/015871.

As used herein the term “pharmaceutical composition” means, for example, a mixture containing a specified amount of a therapeutic compound, e.g. a therapeutically effective amount, of a therapeutic compound in a pharmaceutically acceptable carrier to be administered to a mammal, e.g., a human in order to treat kinase dependent diseases.

As used herein the term “pharmaceutlcally acceptable” refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

The concentration of therapeutic compound in the pharmaceutical composition is present in an amount, e.g. in a therapeutically effective amount, which will depend on absorption, inactivation and excretion rates of the drug as well as other factors known to one of ordinary skill in the art. Furthermore, it is to be noted that dosage values will also vary with the severity of the condition to be alleviated. It is to be further understood that for any particular recipient, specific dosage regimens should be adjusted over time according to the individual need and the professional judgment of the person administering or supervising the administration of the pharmaceutical compositions. The therapeutic compound may be administered once, or may be divided into a number of smaller doses to be administered at varying intervals of time. Thus, an appropriate amount, e.g. an appropriate therapeutically effective amount, is known to one of ordinary skill in the art.

For example, the dose of the therapeutic compound will be in the range from about 0.1 to about 1000 mg per kilogram body weight of the recipient per day. Exemplary unit doses of therapeutic compound range from 100 g to 1000 m, including unit dosages of 100 mg, 200 mg, 300 mg, 400 mg, 600 mg and 800 mg. Alternatively lower doses may be given, for example doses of 0.5 to 100 mg; 0.5 to 50 mg; or 0.5 to 20 mg per kilogram body weight per day. The effective dosage range of the pharmaceutically acceptable salts may be calculated based on the weight of the active moiety to be delivered. If the salt exhibits activity itself, the effective dosage may be estimated as above using the weight of the salt, or by other means known to those skilled in the art.

As used herein the term “immediate-release” refers to the rapid release of the majority of the therapeutic compound, e.g., greater than about 50%, about 55%, about 60%, about 65%, about 70%, about 75%, about 80%, or about 90% within a relatively short time, e.g., within 1 hour, 40 minutes, 30 minutes or 20 minutes after oral ingestion. Particularly useful conditions for immediate-release are release of at least or equal to about 80% of the therapeutic compound within thirty minutes after oral ingestion. The particular immediate-release conditions for a specific therapeutic compound will be recognized or known by one of ordinary skill in the art.

As used herein the term “modified-release” refers to slower release of the majority of the therapeutic compound as compared to immediate release dosage forms, e.g., greater than about 50%, about 55%, about 60%, about 65%, about 70%, about 75%, about 80%, or about 90% within a relatively short time, e.g., within 1 hour, 40 minutes, 30 minutes or 20minutes after oral ingestion. Particularly useful conditions for modified-release are release of at least or equal to about 80% of the therapeutic compound after thirty minutes after oral ingestion. The particular modified-release conditions for a specific therapeutic compound will be recognized or known by one of ordinary skill in the art.

As used herein the term “excipient” refers to a pharmaceutically acceptable ingredient that is commonly used in the pharmaceutical technology for preparing granule and/or solid oral dosage formulations. Examples of categories of excipients include, but are not limited to, binders, disintegrants, lubricants, glidants, stabilizers, fillers and diluents. One of ordinary skill in the art may select one or more of the aforementioned excipients with respect to the particular desired properties of the granule and/or solid oral dosage form by routine experimentation and without any undue burden. The amount of each excipient used may vary within ranges conventional in the art. The following references which are all hereby incorporated by reference disclose techniques and excipients used to formulate oral dosage forms. See The Handbook of Pharmaceutical Excipients, 4^{th} edition, Rowe et a., Eds., American Pharmaceuticals Association (2003); and Remington; the Science and Practice of Pharmacy, 20^{th} edition, Gennaro, Ed., Lippincott Williams & Wilkins (2000).

As used herein, the term “wet granulation” refers to the general process of using a granulation liquid in the granulation process to subsequently form granules, as discussed in Remington: The Science and Practice of Pharmacy, 20^{th} Edition (2000), Chapter 45.

In exemplary embodiments of the present invention, the invented solid dosage forms of nilotinib can be prepared by dry granulation, wet granulation, roller compaction, melt extrusion, spray drying, desolvation, melting followed by rapid solidification and precipitation by solvent-antisolvent processes including supercritical fluids.

The present invention also provides a method of increasing bioavailability by administering the composition or the pharmaceutical composition of the invention, respectively, to an animal or to a patient, wherein the increased bioavailability is determined by comparing the Cmax value or the AUC value of the composition or the pharmaceutical composition of the invention with the composition disclosed in the present invention. Preferably the method increases bioavailability of a drug in administered animal or patient by least 1.3 fold, preferably at least two fold, even more preferably by at least three fold.

In one preferred embodiment of the method, the composition or the pharmaceutical composition of the invention, respectively, comprises 4-methyl-3-[[4-(3-pyridinyl)-2-pyrimidinyl]amino]-N-[5-(4-methyl-1H-imidazol-1-yl)-3-(trifluoromethyl)phenyl] benzamide and the increased bioavailability of nilotinib is least 1.3 fold, preferably at least two fold, even more preferably by at least three fold when compared with 4-methyl-3-[[4-(3-pyridinyl)-2-pyrimidinyl]amino]-N-[5-(4-methyl-1H-imidazol-1-yl)-3-(trifluoromethyl)phenyl] benzamide in the marketed Tasigna™ hard-gelatin capsule manufactured by Novartis.

Bioavailability can be measured by skilled artisan by conventional methods. For example, tablets, capsules, liquids, powders, etc., are given orally to humans or animals and blood levels are measured.

The present invention also provides a method of reducing food effect by administering the composition or the pharmaceutical composition of the invention, respectively, to an animal or to a patient.

“Food effect” in this application is defined as the ratio of the Cmax and/or AUC values of the tested drug in fed dog versus fasted dog. If the ratio is above 1, preferably above 1.1, it is considered the tested drug has food effect. Measuring the Cmax and/or AUC values of the tested drug in fed dog and in fasted dog is standard practice in the art, exemplified by example 2 of the present application. Reduction of food effect can be determined by comparing the value of the ratio from the composition or pharmaceutical composition of the invention and the value of a composition without the solubilized form disclosed in the present invention. Preferably the composition or the pharmaceutical composition of the invention has at least 15% reduced food effect, preferably 20%, preferably 25%, preferably 30%, preferably 40%, reduced food effect.

In one embodiment of the method, the composition comprises solubilized or amorphous 4-methyl-3-[[4-(3-pyridinyl)-2-pyrimidinyl]amino]-N-[5-(4-methyl-1H-imidazol-1-yl)-3-(trifluoromethyl)phenyl] benzamide and having at least 15% reduced food effect, preferably 20%, preferably 25%, preferably 30%, preferably 40%, when compared with 4-methyl-3-[[4-(3-pyridinyl)-2-pyrimidinyl]amino]-N-[5-(4-methyl-1H-imidazol-1-yl)-3-(trifluoromethyl)phenyl] benzamide in a marketed Tasigna™ hard-gelatin capsule manufactured by Novartis and used as the reference product in this invention.

The composition or the pharmaceutical composition according to the invention may also comprise one or more binding agents, filling agents, lubricating agents, suspending agents, sweeteners, flavoring agents, preservatives, buffers, wetting agents, effervescent agents and other excipients. Such excipients are known in the art. Examples of filling agents are lactose monohydrate, lactose anhydrous, microcrystalline cellulose, such as Avicel® PH101 and Avicel® PH102, microcrystalline cellulose and silicified microcrystalline cellulose (ProSolv SMCC®), and various starches; examples of binding agents are various celluloses and cross-linked polyvinylpyrrolidone. Suitable lubricants, including agents that act on the flowabitity of the powder to be compressed, are colloidal silicon dioxide, such as Aerosil® 200, talc, stearic acid, magnesium stearate, calcium stearate and silica gel. Examples of sweeteners are any natural or artificial sweetener, such as sucrose, xylitol, sodium saccharin, cyclamate, aspartame, sucralose, maltitol and acsulfame. Examples of flavoring agents are Magnasweet® (trademark of MAFCO), bubble gum flavor, and fruit flavors, and the like. Examples of preservatives are potassium sorbate, methylparaben, propylparaben, benzoic acid and its salts, other esters of parahydroxybenzoic acid, such as butylparaben; alcohols, such as ethyl or benzyl alcohol. Suitable diluents include pharmaceutically acceptable inert fillers, such as microcrystalline cellulose, lactose, dibasic calcium phosphate, saccharides and/or mixtures of any of the foregoing. Examples of diluents include microcrystalline cellulose, such as Avicel® PH101 and Avicel® PH102; lactose, such as lactose monohydrate, lactose anhydrous, and Pharmatose® DCL21; dibasic calcium phosphate, such as Emcompress®; mannitol; starch; sorbitol; sucrose; and glucose. Examples of effervescent agents are effervescent couples, such as an organic acid and a carbonate or bicarbonate. Suitable organic acids include, e.g., citric, tartaric, malic, fumaric, adipic, succinic and alginic acids and anhydrides and acid salts. Suitable carbonates and bicarbonates include, e.g., sodium carbonate, sodium bicarbonate, potassium carbonate, potassium bicarbonate, magnesium carbonate, sodium glycine carbonate, L-lysine carbonate and arginine carbonate. Alternatively, only the sodium bicarbonate component of the effervescent couple may be present.

In one embodiment, the composition is in a oral solid dosage form or in oral liquid dosage form. The oral liquid dosage form includes solutions, suspensions. The oral solid dosage form includes tablets, pills, capsules, powders. In one embodiment, the solid dosage form is a tablet.

In one aspect, the present invention provides a process of making the composition comprising the steps of blending the pharmaceutical active ingredient, the compound or the small molecule respectively, with the polymer of the invention. The blend can be further processed to form granules by roller compaction, wet granulation, dry granulation etc. The granules may be further processed to form capsules, compressed into tablets or pills.

The following examples are given to illustrate the present invention. It should be understood, however, that the invention is not to be limited to the specific conditions or details described in the examples below. The following examples are illustrative, but do not serve to limit the scope of the invention described herein. The examples are meant only to suggest a method of practicing the present invention.

Quantities of ingredients, represented by percentage by weight of the pharmaceutical composition, used in each example are set forth in the respective tables located after the respective descriptions. For a capsule, when calculating the weight of the pharmaceutical composition (i.e. the capsule fill weight), the weight of the capsule shell itself is excluded from the calculation.

### EXAMPLE 1

### Nilotinib Lactic Acid Formulation

It was surprisingly found that nilotinib had a very high solubility in lactic acid (>600 mg/ml at 65° C.) and could maintain its solubility at intestinal pH in presence of surfactants and/or polymers. Nilotinib solubilized modified release solid dosage forms containing lactic acid were developed. This formulation demonstrated higher bioavailability in both fasted and fed condition compared to FMI, and suppressed the food effect associated with nilotinib. Surfactants and/or polymers were used to prevent the precipitation after solubilized nilotinib is released from the formulation matrix. Due to the liquid nature of lactic acid this formulation matrix is in the liquid form. However, by incorporation of additional suitable excipients, the formulation could be solidified at room temperature. This improved the physical and chemical stability of nilotinib in the formulation. In addition, the solid state also provided the opportunity to modulate the drug release rate.

Examples of nilotinib lactic acid formulations are described in Table 1.

TABLE 1
Nilotinib solubilized formulation containing lactic acid

| Ingredient (mg/dose) | Formulation A | Formulation B |
|---|---|---|
| Nilotinib free base | 100 | 200 |
| Lactic acid | 175 | 350 |
| Poloxamer 407 | 60 | 70 |
| Vitamine TPGS | 50 | 60 |
| HPMC 3 cps | 100 | 150 |
| PEG3350 | 160 | — |
| Total | 645 | 830 |

In these formulations, lactic acid was used to dissolve nilotinib and maintain nilotinib in the liquid/solubilized state. Poloxamer 407 and Vitamin E TPGS polymer and/or surfactant, respectively, were used as precipitation inhibitors and in addition these excipients are also known CYP3A4 & Pg-P inhibitors. The dual function of these polymers is also critical for improving the bioavailability. HPMC 3 cps was used as the control release agent. PEG3350 was used as a solidifying agent to convert the formulation to a solid state at RT.

Manufacturing Process

1. The blend of Poloxamer 407, Vitamin-E TPGS and/or PEG3350 was heated to 65° C. to form a clear solution (solution A).

2. AMN107 free base was dissolved in lactic acid at 65° C. (solution B).

3. Mix solution A and B, and then add HPMC 3 cps to form a suspension

4. The molten suspension was filled in Size 0/00 capsules and allowed to solidify at room temperature.

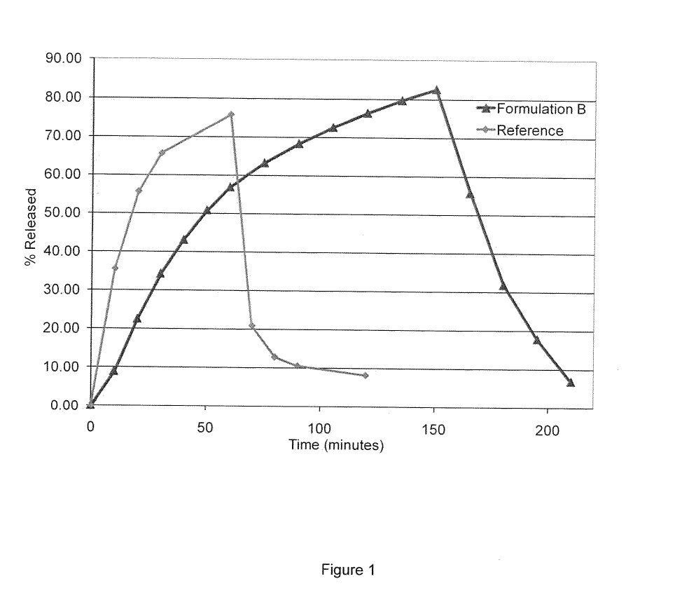

Two step Dissolution: 37° C., 500 ml pH 2 buffer to 1000 ml pH 8.8 buffer. USP Paddle at 75 rpm. It shows that Formulation B is a modified release formulation and nilotinib precipitation could be inhibited after its release from the matrix. In case of the FMI (reference Tasigna capsules), nilotinib precipitates immediately after switching media pH from 2 to 8.8 (FIG. 1).

Dog PK Study

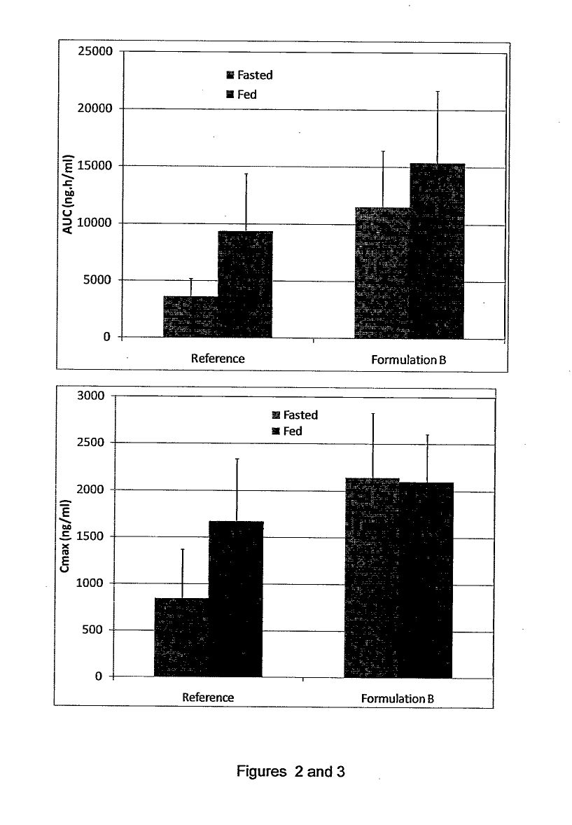

FIGS. 2 and 3 summarize dog PK data (Cmax and AUC) of Formulation B (200 mg nilotinib) in the fasted and fed conditions. This formulation shows higher bioavailability in both fasted and fed conditions in dogs compared to FMI, and suppresses the food effect associated with nilotinib.

Chemical Stability

Nilotinib has demanding purity and stability requirements for a mutagenic impurity 371-03 (<3 ppm at release and <6 ppm during stability). Formulation B exhibits impurity levels of 2.3 ppm at the initial time point, but exhibits impurity levels of 19.4 ppm after 1 month storage at RT, which is over the 6 ppm specification limit. This specification has been set for the FMI. The reason for this increase is due to the high percentage of water content (10% w/w) in the lactic acid. In order to overcome this stability concern, use of pure lactic acid and storage under refrigeration at 2-8° C. is recommended.

### EXAMPLE 2

### Nilotinib Citric Acid Solid Dosage Formulations

In order to overcome this stability issue of lactic acid solubilized nilotinib solid dosage forms, solid organic acids were considered. Surprisingly, citric acid was found to provide remarkably high solubility of the drug in ethanol. This approach allowed development of a proprietary spray drying process as a means to generate solubilized solid dosage form of nilotinib. The resulting AMN107 solubilized drug intermediate was compressed into MR (fast and slow release) tablets with additional external excipients, which showed good chemical stability and also suppressed the food effect in dogs.

Examples of compositions of solubilized solid AMN107 spray dried drug intermediates are described in Table 2.

TABLE 2
Compositions of solubilized
intermediates of nilotinib using citric acid.

| Ingredient | Nilotinib | Nilotinib |
|---|---|---|
| (mg/dose) | intermediate A | intermediate B |
| Nilotinib HCl | 220 | 220 |
| Citric acid, | 300 | 300 |
| anhydrate | | |
| PVP K30 | 200 | 250 |
| Vitamin E | — | 35 |
| TPGS | | |
| Total | 720 | 805 |

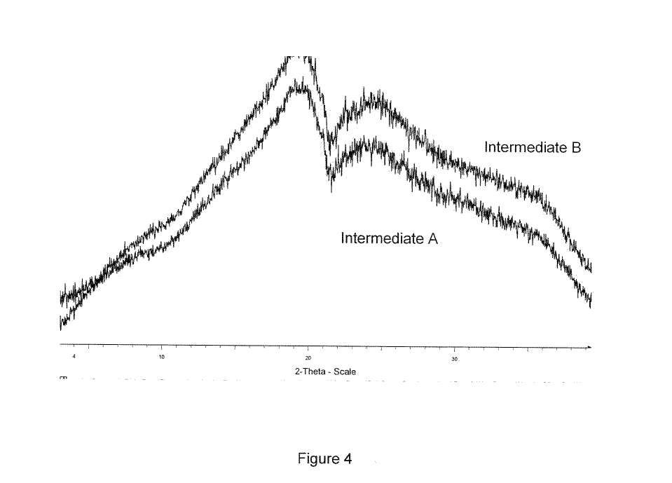

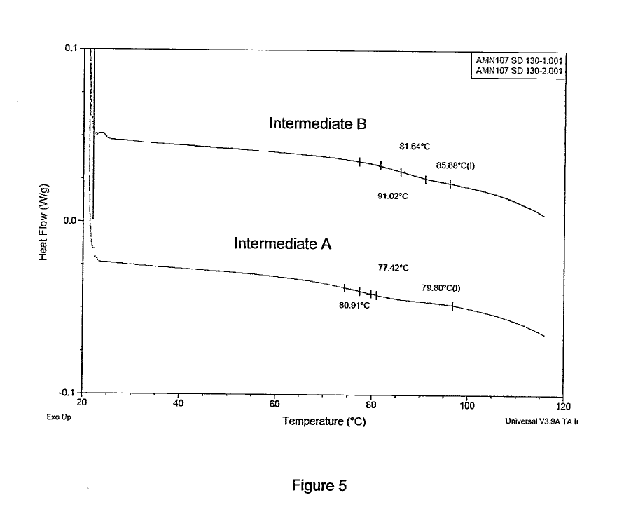

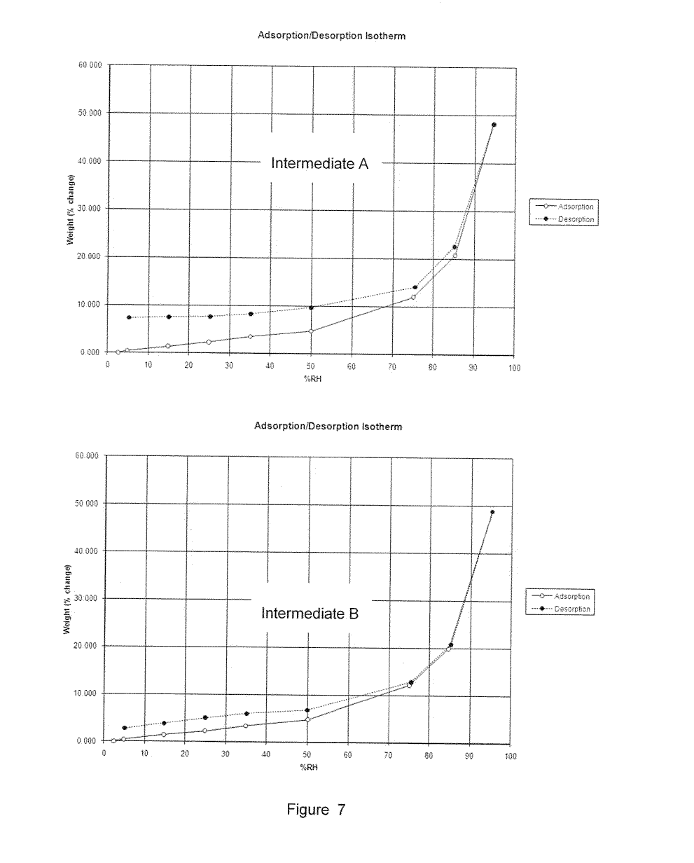

FIGS. 4-7 shows that the Nilotinib intermediate A and B is amorphous with Tg of 77.42° C. and 81.64° C. respectively and can adsorb ˜5% (w/w) water at 25° C. and 50% RH. Intermediate A was mixed with additional external phase excipients and compressed into tablets. Examples of these tablets are described in Table 3. An IR capsule was also included as a reference to compare with the IR tablet to determine the effect of tablet compression.

TABLE 3
Nilotinib immediate released (IR)/modified release
(MR) tablet/capsule compositions (fast and slow
release) containing citric acid as a solubilizing agent.

| Ingredient | MR tablet | IR | IR | MR tablet | MR tablet |
|---|---|---|---|---|---|
| (mg/dose) | A | capsule | tablet | B (fast) | C (slow) |
| Intermediate A | 720 | 720 | 720 | 720 | 720 |
| Poloxamer 407 | 70 | 70 | 70 | 70 | 70 |
| Avicel | 150 | — | — | — | — |
| Crospovidone | — | 50 | 50 | — | — |
| Eudragit L100-55 | — | — | — | 100 | 100 |
| HPMC 3 cps | 50 | — | — | — | — |
| HPMC K100 LV | — | — | — | — | 100 |
| CR | | | | | |
| Aerosil | 10 | 5 | 5 | 5 | 5 |
| Magnesium streate | 5 | 8 | 8 | 8 | 8 |
| Magnesium streate | 5 | — | 4 | — | — |
| (external) | | | | | |
| Total | 1010 | 853 | 857 | 903 | 1003 |
| 50 mg dose | — | 213 | 214 | 225.7 | 250.7 |

Manufacturing Process

MR tablet A, IR capsule and IR tablet were prepared by roller compaction as described in the following steps.

1. All the ingredients except magnesium stearate were passed through No.35 mesh and blended (200 revolutions).

2. Magnesium stearate (internal) was added to step 1 and blended (80 revolutions).

3. The blend was roller compacted.

4. The ribbon was milled and screened through No. 18 mesh.

5. The external magnesium stearate was added to the granules from step 4 and blended (80 revolutions). This final blend was then compressed. For capsule, no external magnesium stearate was added before filling into capsules

MR tablet B (fast) and MR tablet C (slow) were dry blended as described in the following steps.

1. All the ingredients except magnesium stearate were passed through No.35 mesh and blended (200) revolutions.

2. Magnesium stearate was added to step 1 and blended (80 revolutions). The final blend was compressed into tablets.

Chemical Stability

MR tablet A exhibited mutagenic impurity levels of 2.05 ppm at the initial time. After 1 month storage at 40° C. and 75% RH, if exhibited impurity levels of 2.3 ppm in the presence of 1 g desiccant while, impurity levels of 12.8 ppm in the absence of desiccant were observed which is above the specification limits. This data therefore justifies the need for the desiccant for long-term stability.

Physical Stability

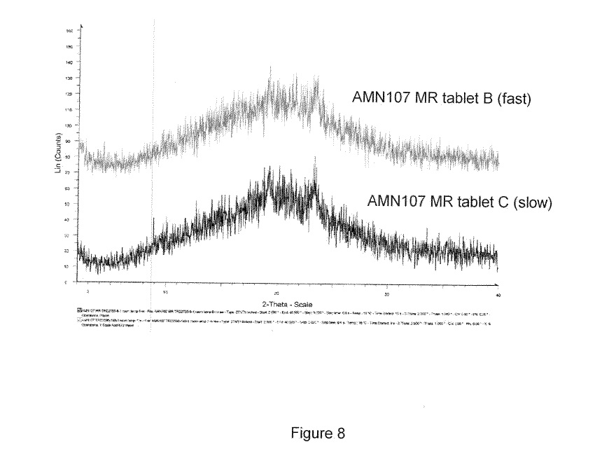

FIG. 8 summarizes XRD of AMN107 MR tablet B and C after 8 months storage at 25° C. and 60% RH. After 6 months under these conditions, AMN107 MR tablet B and C, respectively, maintained their amorphous nature.

Dissolution

Two step dissolution conditions used for the following nilotinib formulations, IR capsule, IR tablet and MR tablet B (fast) are: 37° C.; Step 1, 0-60 minutes 500 ml pH 2 buffer, Step 2, >60 minutes 1000 ml pH 6.8 buffer; Paddle at 75 rpm. Two step dissolution conditions used for MR tablet C (slow) are; 37° C.; Step 1, 0-120 minutes 500 ml pH 2 buffer, Step 2, 120-180 minutes 1000 ml pH 6.8 buffer; Paddle at 75 rpm.

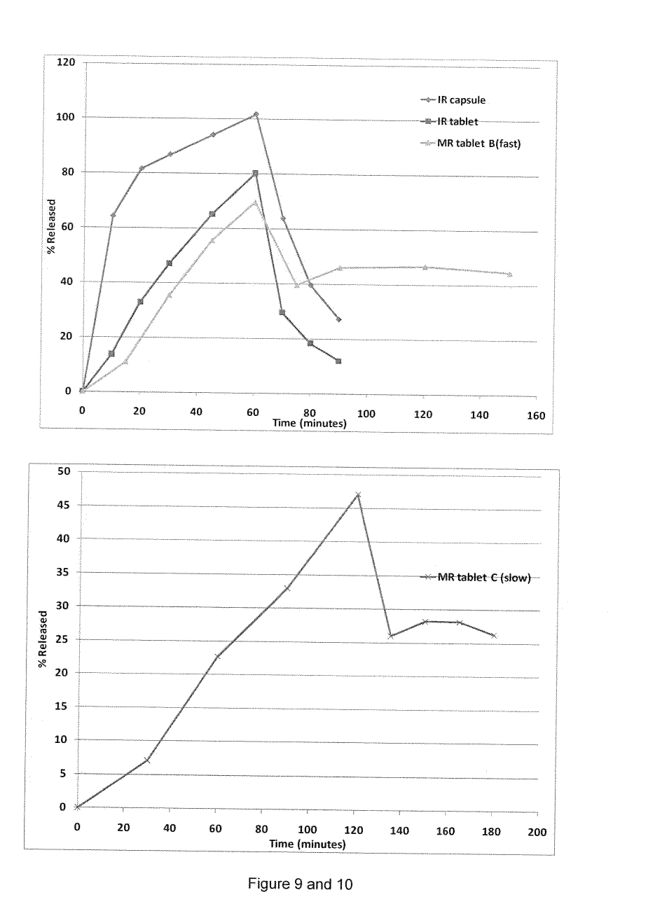

The dissolution data for IR tablet and capsule and MR tablet B (fast) and MR tablet C (slow) are summarized in FIGS. 9 and 10. It can be seen that the IR capsule has a faster dissolution rate compared to the IR tablet. MR Tablet B (fast) containing Eudragit L100-55 shows a slightly slower release rate in pH 2 and higher supersaturation in pH 6.8 compared to IR tablet without Eudragit L100-55. Eudragit L100-55 is an anionic polymer soluble at pH 6.8 and provides inhibition of precipitation. Thus the use of other precipitation inhibitors is expected to provide similar supersaturation. The slow release MR tablet C formulation was developed through screening of several viscosity grade polymers and subsequent selection of an appropriate polymer. The selected polymer, HPMC K100 LV CR had the required viscosity and provided the expected release profile. As can be seen from FIG. 10, the MR tablet C (slow) containing Eudragit L100-55 and HPMC K100 LV CR demonstrated a slow release in pH 2.

Dog PK Data

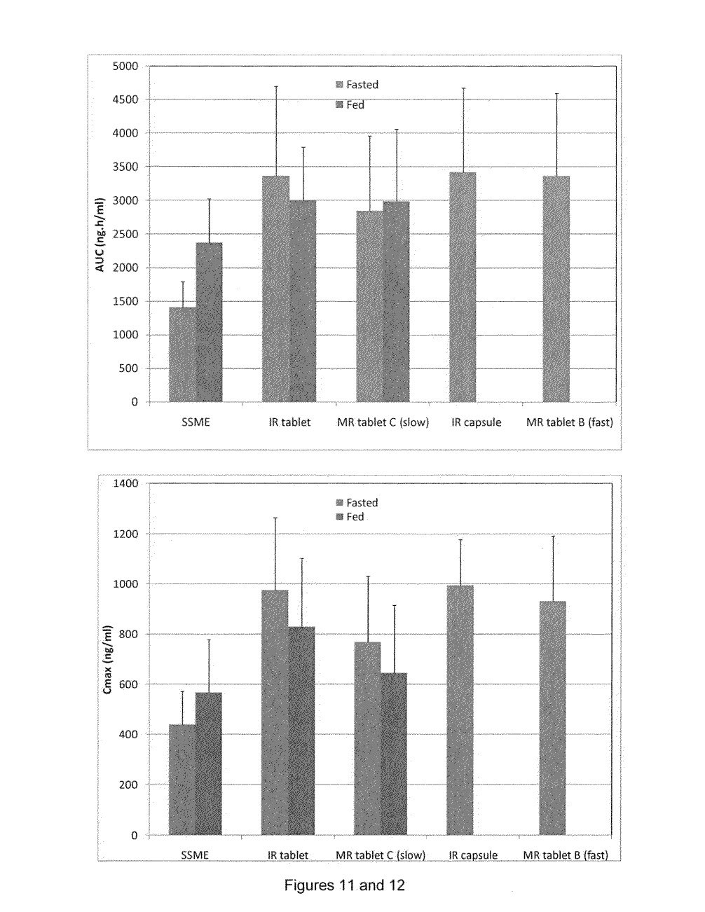

50 mg Nilotinib MR (fast & slow) formulations solubilized using citric acid were tested in dogs. The solid-Suspeneded MicroEmulsion (SSME) formulation previously tested in the clinic was used as the control since it showed a higher bioavailability and moderate suppression of food effect in the human clinical study compared to FMI and thus was deemed to be a better reference to be compared with. The results (FIGS. 11 and 12) show that both IR and MR tablet exhibited enhanced nilotinib bioavailability under both fasted and fed conditions in dogs. As a result, both IR and MR (slow release) nilotinib formulations exhibited no food effects.

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

1. An amorphous 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof.

2. A dosage form comprising amorphous 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof.

3. A dosage form of claim 2 comprising 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and at least one organic acid.

4. A dosage form of claim 2 comprising 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and at least one organic acid, having a fasted state bioavailability that exceeds 130% of a hard-gelatin capsule comprising 4-Methyl-3-[[4-(3-pyridinyl)-2-pyrimidinyl]amino]-N-[5-(4-methyl-1H-imidazol-1-yl)-3-(trifluoromethyl)phenyl] benzamide.

5. A dosage form of claim 1 comprising 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and at least one organic acid, having a fed/fasted ratio of 0.8-1.5 for AUC and/or Cₘₐₓ.

6. The dosage form of claim 3, wherein said at least one organic acid is selected from acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, glutamic acid, aspartic acid, maleic acid, hydroxymaleic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid and ascorbic acid.

7. The dosage form of claim 3, wherein the organic acid is citric acid.

8. The dosage form of claim 3, wherein the organic acid is lactic acid.

9. The dosage form of claim 3, wherein the organic acid is acetic acid.

10. The dosage form of claim 3 further comprising a surfactant or an anionic polymer.

11. The dosage form of claim 10, wherein the surfactant or the anionic polymer is CYP3A4 or Pg-P inhibitor.

12. The dosage form of claim 10, wherein the surfactant is Poloxamer 407 and/or Vitamin E TPGS.

13. The dosage form of claim 10, wherein the polymer is Eudragid L100-55.

14. The dosage form of claim 1, wherein the dosage form has water content of less than 10% w/w, preferably less than 5% w/w, particularly less than 2% w/w.

15. The dosage form of claim 1 further comprising excipients for solidifying the dosage form.

16. The dosage form of claim 1, wherein the dosage form is solid.

17. The dosage form of claim 16, wherein the dosage form is a tablet.

18. The dosage form of claim 16, wherein the dosage form is a capsule.

19. A method for preparing amorphous 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof, comprising the step of adding at least one organic acid.

20. A method for preparing a dosage form comprising amorphous 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and at least one organic acid, comprising the step of melt extruding 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and the at least one organic acid.

21. A method of claim 20, wherein 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and at least one organic acid are mixed and melt extruded together.

22. A method of preparing a dosage form comprising 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and at least one organic acid comprising the step of spray drying at least partly dissolved of 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and adding the at least one organic acid.

23. The method of claim 22, wherein the 4-Methyl-3-[[4-(3-pyridinyl)-2-pyrimidinyl]amino]-N-[5-(4-methyl-1H-imidazol-1-yl)-3-(trifluoromethyl)phenyl] benzamide or a pharmaceutically acceptable salt thereof and the at least one organic acid together are in a solution or suspension for spray drying.

24. The method of claim 20 further comprising a step of adding a surfactant or an anionic polymer.

25. The method of claim 24, wherein the surfactant or the anionic polymer is CYP3A4 or Pg-P inhibitor.

26. The method of claim 24, wherein the surfactant is Poloxamer 407 and/or Vitamin E TPGS.

27. The method of claim 24, wherein the polymer is Eudragid L100-55.

28. The method of claim 20 comprising a further step of obtaining a solid dosage form.

29. The method of claim 28, wherein the solid dosage form is a tablet or a capsule.

30. Use of organic acid for increasing of bioavailability of nilotinib.

31. Use of organic acid for supressing the food effect associated with pharmaceutical composition comprising nilotinib or a pharmaceutically acceptable salt thereof.

32. A dosage form of claim 1 for use as a medicine.

33. The dosage form of claim 32, wherein the medicine is stored under refrigeration at 2 to 8° C.
